(19) United States
(12) Patent Application Publication
(10) Pub. No.: US 2010/0056646 A1
(43) Pub. Date: Mar. 4, 2010

(54) Hydroswellable, segmented, aliphatic polyurethane ureas and intra-articular devices therefrom

(76) Inventors: Shalaby W. Shalaby, Anderson, SC (US); Georgios T. Hilas, Anderson, SC (US)

Correspondence Address: LEIGH P. GREGORY, PO BOX 168, CLEMSON, SC, 29633-0168, US

(21) Appl. No.: 12/586,788

(22) Filed: Sep. 28, 2009

Related U.S. Application Data
(63) Continuation-in-part of application No. 12/380,391, filed on Feb. 26, 2009.
(60) Provisional application No. 61/069,046, filed on Mar. 12, 2008.

(51) Int. Cl. A61K 47/30; C08G 18/00; C08G 18/48; C08G 77/04; B05D 3/00

(52) U.S. Cl. 514/772.3; 528/44; 528/76; 528/28; 427/2.26

(57) ABSTRACT

Hydroswellable, non-absorbable, biostable, segmented, aliphatic polyether-urethane-ureas or polyether-siloxane-urethane-ureas form a single component, intra-articular device for restoring joints with artificial cartilage, as a cartilage substitute for degenerated cartilage and/or for enhancing the remaining cartilage of an arthritic joint. The intra-articular devices can be bicomponent in nature comprising a biostable, articulating, non-absorbable component and an absorbable component in the form of a solid or microporous liner interfacing with the tissue of defective or diseased joint to support in situ tissue engineering. One or more bioactive agent with specific pharmacological function can be incorporated in the single or bicomponent intra-articular devices to supplement their structural functions.

This present application is a continuation in part of U.S. Ser. No. 12/380,391 filed on Feb. 26, 2009, which claims the benefit of prior provisional application, U.S. Ser. No. 61/069,046 filed on Mar. 12, 2008.

### FIELD OF THE INVENTION

This invention is directed to the use of hydroswellable (or water-swellable) absorbable and/or non-absorbable segmented aliphatic polyether-urethane ureas to form intra-articular devices for restoring joints with artificial cartilage as a cartilage substitute for degenerated cartilage, enhancing the remaining cartilage of an arthritic joint, and/or supporting in situ cartilage tissue engineering.

### BACKGROUND OF THE INVENTION

The parent application (U.S. Ser. No. 12/380,391) is directed in part to (1) the design and synthesis of film-forming hydroswellable, non-absorbable or absorbable segmented, aliphatic polyether-urethane-ureas, comprising polyoxyalkylene chains which may be covalently linked to a second group of chain segments selected from the group of polyalkylene carbonate chains and polyester chains, the second group of chain segments interlinked with a third group of chain segments, the third chain segments selected from the group consisting of aliphatic urethane segments and aliphatic urea segments, the composition exhibiting at least three (3) percent increase in volume when placed in the biological environment where it maintains its initial physicochemical properties or undergoes changes at predetermined rates, depending on the composition of the constituent segments; (2) non-absorbable polyether-siloxane-urethane-ureas; (3) incorporating bioactive agents to augment the polymer function as an intra-articular device; and (4) the use of selected members of absorbable polyester-ester-urethane as rheology modifier of absorbable cyanoacrylate-based tissue adhesives. However, the parent patent application was silent as to the practical, specific uses of any of the materials described in items 1 through 4 above. Accordingly, this invention is directed toward the integrated use of selected polymeric and monomeric components of the parent application to form and use intra-articular devices for restoring joints with artificial cartilage as a cartilage substitute for degenerated cartilage and enhancing the remaining cartilage of an articular joint and/or supporting cartilage tissue engineering.

### SUMMARY OF THE INVENTION

Generally, the present invention is directed to a solution comprising a hydroswellable, segmented, aliphatic, polyurethane composition in a water-soluble aliphatic solvent, having a boiling point of less than 90° C. at atmospheric pressure, capable of film formation by casting and microfiber formation by electrostatic spinning at room temperature, wherein the solvent comprises at least one member selected from the group consisting of trifluoroethanol, hexafluoroisopropyl alcohol and higher homologs, and wherein the polyurethane composition comprises at least one member selected from the group consisting of polyether-urethane-urea, polyether-dimethylsiloxane-urethane-urea and polyether-ester-urethane-urea, and further wherein the polyurethane composition comprises a polyether-urethane-urea dissolved in trifluoroethanol.

A technological aspect of this invention is directed to a method for making a film which includes the steps of forming a solution comprising a hydroswellable, segmented, aliphatic, polyurethane composition in a water-soluble aliphatic solvent, having a boiling point of less than 90° C. at atmospheric pressure, and a multistep casting of the solution at room temperature to form a uniform film having a thickness of at least 0.5 mm, wherein the polyurethane composition comprises a polyether-urethane-urea dissolved in trifluoroethanol, and wherein the film formation takes place on the surface of a mold form or template having a peripheral geometry similar to that of a head of a bone for an articulating joint, and further wherein the head of a bone is that of a femur and the formed film is in the shape of a femoral cap extending into a collar or sleeve covering the proximal end of the femur stem. Additionally, the collar is reinforced with knitted or woven fabric comprising absorbable or non-absorbable fibers wherein the fabric-reinforced collar or sleeve is anchored to the bone stem using a member selected from the group consisting of absorbable tissue adhesive absorbable tacks, non-absorbable staples and absorbable staples.

Another technological aspect of the instant invention is directed to a method for making a film which includes the steps of forming a solution comprising a hydroswellable, segmented, aliphatic, polyurethane composition in a water-soluble aliphatic solvent, the polyurethane composition comprising an absorbable polyether-ester-urethane-urea and the solvent comprising trifluoro-ethanol, the solution having a boiling point of less than 90° C. at atmospheric pressure, and casting the solution at room temperature to form a uniform film having a thickness of at least 0.5 mm onto a mold form simulating a femur bone head and proximal end of its stem, wherein the formed film is in the form of a thin, absorbable liner component of the cap/collar combination for a femur. Additionally, a non-absorbable polyurethane composition in trifluoroethanol is cast onto said absorbable liner thereby forming a 2-component intra-articular device comprising a non-absorbable articulating component and an absorbable liner component for intimate contact with defective or diseased femur bone, wherein the collar is reinforced with knitted or woven fabric comprising absorbable or non-absorbable fibers and wherein the fabric-reinforced collar or sleeve is anchored to the bone stem using a member selected from the group consisting of absorbable tissue adhesive absorbable tacks, non-absorbable staples and absorbable staples. Alternatively, (a) the non-absorbable component of the 2-component intra-articular device comprises at least one member of the group consisting of polyether-urethane-urea and polyetherdimethylsiloxane-urethane-urea and (b) the absorbable component of the cap/collar combination is limited mostly to the cap part of the said combination.

A clinical aspect of this invention is directed to a solution of a hydroswellable, segmented, aliphatic, polyurethane composition in a water-soluble aliphatic solvent, having a boiling point of less than 90° C. at atmospheric pressure, capable of film formation by casting and microfiber formation by electrostatic spinning at room temperature wherein said polyurethane composition further comprises at least one bioactive agent.

### DETAILED DESCRIPTION OF PREFERRED EMBODIMENTS

The present invention is directed, primarily, to rendering a family of hydroswellable polyurethanes exceptionally useful, clinically, toward the formation of intra-articular devices for restoring joints with artificial cartilage as a cartilage substitute for degenerated cartilage, enhancing the remaining cartilage of an arthritic joint, and/or supporting in situ cartilage tissue engineering. Subjects of the instant invention are physicochemical means needed for achieving these different forms of artificial cartilages. These include (1) designing the aliphatic polyether-urethane urea molecular chain to yield hydroswellable material with exceptional mechanical properties, increased degrees of swelling in the biological environment through having sufficiently high molecular weight chains with highly hydrophilic segments and biostability to ensure prolonged performance at the biological site; (2) modifying the composition of the polyether-urethane ureas noted in item 1 to render them soluble in volatile solvents, which facilitate the application during the conversion of the polymer solution into uniform hydroswellable film on a mold form corresponding to the peripheral geometry of a joint, thus simulating the actual joint under non-destructive, controllable and mild conditions as is the case of multistep solution casting and/or electrospinning; (3) incorporating in the molecular chain of the polyether-urethane urea a polydimethylsiloxane segment to increase the polymer hydrolytic stability and hence, the device stability in the biological environment for prolonged functional performance; (4) devising means to apply the polymers of items 1 through 3 at a controllable rate on a mold form (template or scaffold) simulating the femoral head of humans or animals using a solvent unique to those used in traditional polyurethane casting technology, which needs to boil below 90° C., have high vapor pressure at room temperature to allow its use under ambient conditions and to be water-soluble to allow effective removal of residual solvent from the shaped articles, such as femoral caps; (5) designing a mold form (template or scaffold) simulating the femoral head and its proximal end to facilitate its deployment onto the natural bone and its position retention—at the application site using a knitted or woven sleeve to reinforce the collar segment of the device, interfacing with the proximate end of the femur stem—the fabric reinforcement can be constructed from absorbable or non-absorbable yarns and the reinforced sleeve can be further anchored to the femur stem using absorbable or non-absorbable tacks or staples and/or absorbable cyanoacrylate-based tissue adhesives; (6) designing the device, as in a femoral cap, with an absorbable luminal (inner) liner that apposes the natural tissue and allows new tissue regeneration that parallels the hydrolytic degradation and mass loss of the absorbable liner, which can vary in thickness, density and porosity depending on the application site and specific clinical situation—the absorbable liner can be first formed on the mold form prior to forming the non-absorbable articulating component of the intra-articular cartilage; and (7) designing the physicochemical morphological properties of the absorbable liner in item 6 to exhibit controlled absorption, mass loss and strength retention profiles as well as variable levels of thickness and porosity—the absorbable liner can be in the form of a flexible, solid film, electrostatically spun microfibrous fabric, microporous film which is made by first depositing a solid film reinforced with a methylene chloride-soluble absorbable mesh which can be dissolved leaving behind the continuous microporous film upon which the non-absorbable film can be deposited. Furthermore, the intra-articular device described above in items 1 through 7 can be made as a cap having the peripheral geometry similar to that of a head of a bone for a joint to be restored. Included in said bones are the human or animal femur and tibia. Besides the hip and knee joints, the material of the intra-articular device can be used more generally for restoring other types of diseased or defective articulating joints in humans or animals.

One or more bioactive agent can be incorporated in the non-absorbable and/or absorbable component of the intra-articular devices to supplement their structural functions. These bioactive agents can belong to those known to (1) accelerate cartilage or bone growth; (2) exhibit antimicrobial activities; and (3) display anti-inflammatory activities. Other bioactive agents known for other specific pharmacological activities can be incorporated in the single component device and in one or both components of the bicomponent intra-articular devices.

Further illustrations of the present invention are provided by the following examples:

### Example 1

### Synthesis and Characterization of a Biostable Polyether-urethane-urea (PEUU)

To prepare a biostable Polyether-urethane-urea (PEUU) the following steps were pursued. Poly(ethylene glycol)-block-poly(propylene glycol)-block-poly(ethylene glycol) (PEG-PPG-PEG, Mₙ=14.6 kDa) (72.0 grams, 0.0049315 moles) and poly(tetramethylene glycol) (PTMG, Mₙ=2.9 kDa) (168.0 grams, 0.057931 moles) were weighed out and placed into a 2.0 liter glass reaction kettle. The PEG-PPG-PEG and PTMG were placed under vacuum (<0.5 mmHg) and dried at 140° C. for three hours. The remainder of the synthesis was carried out under a nitrogen blanket. The kettle was then brought to a temperature of 60° C. and N,N-dimethylacetamide (DMAC) (560 mL) was added into the reaction kettle to make a 30 wt. % solution in DMAC. The reaction mixture was stirred for one hour at 60 revolutions per minute (rpms) and then for another hour at 100 rpms in order to fully dissolve. Once dissolved the reaction temperature was lowered to room temperature and the mixing speed was increased to 200 rpms. 1-6-diisocyanatohexane (15.86 grams, 0.094294 moles) was then added to the reaction kettle in four aliquots using a 5000 microliter pipette. Following two hours of stirring SnOct (5.92 mL of a 0.2M solution in 1,4-dioxane, 0.0011849 moles) was added and the reaction mixture was stirred at 200 rpms for an additional 15 minutes. The kettle temperature was then increased to 100° C. and the mixing speed was lowered to 120 rpms. These reaction parameters were held for two hours after which the kettle was cooled back down to room temperature. Ethylene diamine (EtDA) was added to the reaction using the following process. EtDA (1.889 grams, 0.031431 moles) was weighed into 30 mL of DMAC. This EtDA solution was then added to the reaction kettle while mixing at 200 rpms over a 30 second period.

The polymer synthesized above was purified by washing in water, washing in acetone, and then finally drying under reduced pressure to a constant weight. Polymer characterization was conducted via inherent viscosity and polymer properties were assessed by film burst testing and swell testing. The results are set forth in the table below.

PEUU Polymer Properties and Characterization

| MTS Burst Testing (0.7 mm thick film) | | | | | |
|---|---|---|---|---|---|
| | | WET^{a} | DRY | | |
| WET^{a} | DRY | Extension | Extension | | |
| Peak | Peak | at Max | at Max | Inherent | |
| Load | Load | Load | Load | Viscosity | Swell^{b} |
| (N) | (N) | (mm) | (mm) | (dL/g) | (% add on) |
| 134 | 160 | 63.42 | 54.84 | 4.55 | 64.8 |

^{a}Placed in water for at least 24 hours prior to testing.
^{b}Placed in 1% methyl cellulose solution for >16 hours.

### Example 2

### Formation and Characterization of an Intra-Articular Device as a Cartilage Substitute for a Sheep Femoral Cap (FC-1) Using PEUU from Example 1

To prepare an intra-articular device as a cartilage substitute for a sheep femoral cap the following steps were pursued. A Teflon femoral cap model was machined using a CNC Machine to the exact dimensions required for the Sheep Femoral Cap (FC-1) device. This Teflon FC-1 model was used as a scaffold for solution casting of the device. The solution casting of the femoral cap device was carried out as follows. A 6% (wt/vol) PEUU (prepared in Example 1) in 2,2,2-trifluoroethanol (TFE) was prepared in a 4 oz. glass jar. The Teflon FC-1 scaffold was slowly lowered into the PEUU solution casting solution to a depth ˜0.5 in. up from the base of the cap and slowly removed. Once removed from the solution the device was secured to a rotation device rotating at ˜6 revolutions per minute in a fume hood. The device was allowed to dry while rotating for 30 minutes before conducting another dip using the same procedure. A total of 29 dips were conducted. After the final dip coating the device was allowed to dry under a fume hood for 16 hours. The solution-cast FC-1 device was then carefully removed from the Teflon FC-1 scaffold. The FC-1 device had a weight of 1.40 grams and an average thickness of 0.97 mm.

### Example 3

### Synthesis and Characterization of a Biostable Polyether-dimethylsiloxane-urethane-urea (PESiUU)

To prepare a Polyether-dimethylsiloxane-urethane-urea (PESiUU) the following steps were pursued. Poly(ethylene glycol)-block-poly(propylene glycol)-block-poly(ethylene glycol) (PEG-PPG-PEG, Mₙ=14.6 kDa) (36.0 grams, 0.0024658 moles) and poly(tetramethylene glycol) (PTMG, Mₙ=2,900 Da) (67.2 grams, 0.0231724 moles) were weighed out and placed into a 1.0 liter stainless steel reaction kettle. The PEG-PPG-PEG and PTMG were placed under vacuum (<0.5 mmHg) and dried at 140° C. for three hours. The kettle was then purged with nitrogen and brought to a temperature of 80° C. Poly(dimethylsiloxane), hydroxypropylether terminated (PDMS, Mₙ=1.125 kDa) (16.8 grams, 0.0149333 moles) was added to the reaction kettle and placed back under vacuum (<0.5 mmHg) for 1.5 hours at 80° C. The remainder of the synthesis was carried out under a nitrogen blanket. N,N-dimethylacetamide (DMAC) (280 mL) was added into the reaction kettle to make a 30 wt. % solution in DMAC. The reaction mixture was stirred for one hour at 60 revolutions per minute (rpms) and then for another hour at 100 rpms in order to fully dissolve. Once dissolved the reaction temperature was lowered to 65° C. and the mixing speed was increased to 170 rpms. 1-6-diisocyanatohexane (10.24 grams, 0.060857 moles) was added to the reaction kettle in two aliquots using a 5000 microliter pipette. Following two hours of stirring SnOct (2.965 mL of a 0.2M solution in 1,4-dioxane, 0.0005924 moles) was added and the reaction mixture was stirred at 170 rpms for an additional 30 minutes. The kettle temperature was then increased to 100° C. and the mixing speed was lowered to 120 rpms. These reaction parameters were held for two hours after which the kettle was cooled back down to room temperature. Ethylene diamine (EtDA) was added to the reaction using the following process. EtDA (1.219 grams, 0.020286 moles) was weighted out into 20 mL of DMAC. This EtDA solution was then added to the reaction kettle while mixing at 200 rpms over a 30 second period.

The polymer was purified by washing in water, washing in acetone, and then finally drying under reduced pressure to a constant weight before characterization by inherent viscosity as in Example 1.

### Example 4

### Formation and Characterization of an Intra-Articular Device as a Cartilage Substitute for a Sheep Femoral Cap (FC-2) Using PESiUU from Example 3

To prepare an intra-articular device as a cartilage substitute for a sheep femoral cap using PESiUU the procedure documented in Example 2 was followed with the use of PESiUU polymer in the casting solution instead of PEUU.

### Example 5

### Formation of a Sleeved Fiber-Reinforced Biostable Femoral Cap (R-FC-1) Based on FC-1 of Example 2

To prepare a sleeved fiber-reinforced biostable femoral cap (R-FC-1) the following steps were pursued. A circular mesh was knitted using a weft knitting pattern out of a multifilament polyethylene terephthalate yarn with a 0.865 inch diameter knitting head. This mesh was then placed on a Teflon FC-1 model (prepared in Example 2). A 6% (wt/vol) PEUU (prepared in Example 1) solution in 2,2,2-trifluoroethanol (TFE) was prepared in a 4 oz. glass jar. The Teflon FC-1 scaffold containing the mesh was slowly lowered into the PEUU casting solution to a depth ˜0.5 in. up from the base of the cap and slowly removed. Once removed from the solution the device was secured to a rotation device rotating at ˜6 revolutions per minute in a fume hood. The device was allowed to dry while rotating for 30 minutes before conducting another dip using the same procedure. A total of 7 dips were conducted. After the final dip coating the meshed device was allowed to dry under a fume hood for 16 hours. The PEUU coated mesh sleeve was then carefully removed from the Teflon FC-1 scaffold and trimmed in such a way that when replaced onto the Teflon FC-1 scaffold it would only cover the non-articulating portions of FC-1 and form a sleeve around the bottom portion of the cap. The trimmed PEUU coated mesh sleeve was then removed from the Teflon FC-1 scaffold and set aside. The Teflon FC-1 model was dip coated with a total of 29 dips as previously reported in Example 2 with the following changes: immediately following the 6^{th} dip coating the trimmed PEUU coated mesh sleeve was carefully placed onto the device. The solution-cast R-FC-1 device was then carefully removed from the Teflon FC-1 scaffold.

### Example 6

### Formation of a Sleeved Fiber-Reinforced Biostable Femoral Cap (R-FC-2) based on FC-2 of Example 4

To prepare a sleeved fiber-reinforced biostable femoral cap (R-FC-2) the procedure documented in Example 5 was followed with the use of PESiUU polymer in the casting solution instead of PEUU.

### Example 7

### Synthesis and Characterization of an Absorbable Polyether-ester-urethane-urea (PEEUU)

To prepare an absorbable Polyether-ester-urethane-urea (PEEUU) the following steps were pursued. A PEEUU pre-polymer consisting of a polylactide terminated poly(tetramethylene glycol) was first prepared using the following procedure. Poly(tetramethylene glycol) (PTMG, Mₙ=2.9 kDa) (70.0 grams, 0.024138 moles) was weighed out and placed into a 250 mL two-neck round bottom reaction flask. The reaction flask was placed under vacuum (<0.5 mmHg) and dried for three hours at 140° C. The temperature was lowered to 80° C. and the reaction flask was purged with nitrogen. Once the reaction flask was purged with nitrogen, dl-lactide (30.0 grams, 0.20833 moles) was added and stirred at 60 revolutions per minute (rpms) for 30 minutes at 80° C. The temperature was increased to 110° C. and the stir rate was increased to 140 rpms. After one hour SnOct (0.233 mL of a 0.2M solution in toluene, 0.000046494 moles) was added to the reaction and the temperature was increased to 160° C. These reaction conditions were maintained for 2 hours.

PEEUU pre-polymer (40.0 grams, 0.0096548 moles), synthesized above, was weighed out and placed into a 250 mL two-neck round bottom reaction flask and placed under vacuum (<0.5 mmHg) at 100° C. for three hours. The reaction temperature was reduced to 65° C. and N,N-dimethylacetamide (DMAC) (94.0 mL) was added to the reaction. The reaction mixture was stirred at a rate of 140 rpms for 2 hours. The stirrer rate was then increased to 170 rpms and 1-6-diisocyanatohexane (2.44 grams, 0.0144823 moles) was added. After one hour of stirring at 170 rpms, SnOct (0.990 mL of a 0.2M solution in 1,4-dioxane, 0.00019896 moles) was added. The reaction temperature was increased to 100° C. and the stir rate was lowered to 120 rpms. These reaction conditions were held for 3 hours after which the reaction flask was cooled down to room temperature. Ethylene diamine (EtDA) was added to the reaction using the following process. EtDA (0.292 grams, 0.004864 moles) was weighed out into 10 mL of DMAC. This EtDA solution was then added to the reaction kettle while mixing at 200 rpms over a 10 second period. Following EtDA addition, stir rate was lowered to 60 rpms and continued for 30 minutes. Purification was carried out by blending polymer produced above with ice water followed by drying under vacuum to a constant weight. Characterization is conducted via inherent viscosity.

### Example 8

### Formation of a 2-Component, Fiber-Reinforced Sleeved Femoral Cap (2-FC) Using a Biostable PEUU (from Example 1) and an Absorbable PEEUU Liner Film (from Example 7)

A femoral cap, 2-FC, using a biostable PEUU (from Example 1) and an absorbable PEEUU (from Example 7) is made according to the following these steps. A PEUU coated mesh sleeve is made as in Example 5. A 6% (wt/vol) PEEUU solution in 2,2,2-trifluoroethanol (TFE) and a 6% (wt/vol) PEUU solution in TFE are prepared in 4 oz. glass jars. The Teflon FC-1 scaffold (prepared in Example 2) is slowly lowered into the PEEUU casting solution to a depth ˜0.5 in. up from the base of the cap and slowly removed. Once removed from the solution the device is secured to a rotation device rotating at ˜6 revolutions per minute in a fume hood. The device is allowed to dry while rotating for 30 minutes before conducting another dip using the same procedure. A total of 6 dips are conducted using the absorbable PEEUU casting solution. Immediately following the final dip in the PEEUU solution, the PEUU coated mesh sleeve is carefully placed onto the device. Another 23 dip coatings are conducted using the PEUU casting solution. After the final dip coating the meshed device is allowed to dry under a fume hood for 16 hours. The fiber-reinforced sleeved femoral cap (2-FC), using a biostable PEUU and an absorbable PEEUU liner film, is then carefully removed from the Teflon FC-2 scaffold.

### Example 9

### Formation of a 2-Component Femoral Cap (2-FC) Using a Biostable PEUU (from Example 1) and an Absorbable PEEUU Microporous Liner from Film (from Example 7)

Formation 2-FC with microporous 2-FC is conducted as described in Example 8 with the exception of applying the absorbable component (a) by electrospinning to form microfibrous, non-woven fabric, and (b) spraying the absorbable polymer on a reinforced mesh of poly-caprolactone, which later is extracted with methylene chloride leaving behind a microporous liner.

### Example 10

### Preparation and Evaluation of an Absorbable Cyanoacrylate Tissue Adhesive (HAT-1)

This was made by mixing an 80/20 methoxypropyl cyanoacrylate/ethyl cyanoacrylate solution with a stabilizer against premature anionic polymerization and a rheology-modifier from Example 7. The formulation was tested for viscosity and joint adhesive strength to verify its effective use as a tissue adhesive for anchoring the sleeved cap onto a femoral bone.

### Example 11

### In Vitro Anchoring of Sleeved Cap from Example 5 to a Femoral Bone Using Tissue Adhesive Formulation from Example 10

To anchor the sleeved femoral cap R-FC-1 device to a femoral bone using tissue adhesive formulation HAT-1 the following procedure is followed. Immediately prior to application of the R-FC-1 device to the femoral condyle, a small amount of HAT-1 is applied to the bone adhesion site and to the inside surface of the R-FC-1 femoral cap. The adhesive joint is allowed to form as the monomer cures in about one minute.

Although the present invention has been described in connection with the preferred embodiments, it is to be understood that modifications and variations may be utilized without departing from the principles and scope of the invention, as those skilled in the art will readily understand. Accordingly, such modifications may be practiced within the scope of the following claims. Moreover, Applicant hereby discloses all subranges of all ranges disclosed herein. These subranges are also useful in carrying out the present invention.

What is claimed is:

1. A solution comprising a hydroswellable, segmented, aliphatic, polyurethane composition in a water-soluble aliphatic solvent, having a boiling point of less than 90° C. at atmospheric pressure, capable of film formation by casting and microfiber formation by electrostatic spinning at room temperature.

2. A solution as in claim 1 wherein the solvent comprises at least one member selected from the group consisting of trifluoroethanol, hexafluoroisopropyl alcohol and higher homologs.

3. A solution as in claim 1 wherein the polyurethane composition comprises at least one member selected from the group consisting of polyether-urethane-urea, polyether-dimethylsiloxane-urethane-urea and polyether-ester-urethane-urea.

4. A solution as in claim 3 wherein the polyurethane composition comprises a polyether-urethane-urea dissolved in trifluoroethanol.

5. A method for making a film comprising the steps of
forming a solution comprising a hydroswellable, segmented, aliphatic, polyurethane composition in a water-soluble aliphatic solvent, having a boiling point of less than 90° C. at atmospheric pressure, and
casting the solution at room temperature to form a uniform film having a thickness of at least 0.5 mm.

6. The method set forth in claim 5 wherein the polyurethane composition comprises a polyether-urethane-urea dissolved in trifluoroethanol.

7. The method set forth in claim 5 wherein the film formation takes place on the surface of a mold form or template having a peripheral geometry similar to that of a head of a bone for an articulating joint.

8. The method set forth in claim 7 wherein the head of a bone is that of a femur and the formed film is in the shape of a femoral cap extending into a collar or sleeve covering the proximal end of the femur stem.

9. The method set forth in claim 8 wherein the collar is reinforced with knitted or woven fabric comprising absorbable or non-absorbable fibers.

10. The method set forth in claim 9 wherein the fabric-reinforced collar or sleeve is anchored to the bone stem using a member selected from the group consisting of absorbable tissue adhesive absorbable tacks, non-absorbable staples and absorbable staples.

11. A method for making a film comprising the steps of
forming a solution comprising a hydroswellable, segmented, aliphatic, polyurethane composition in a water-soluble aliphatic solvent, the polyurethane composition comprising an absorbable polyether-ester-urethane-urea and the solvent comprising trifluoroethanol, the solution having a boiling point of less than 90° C. at atmospheric pressure, and
casting the solution at room temperature to form a uniform film having a thickness of at least 0.5 mm onto a mold form simulating a femur bone head and proximal end of its stem.

12. The method set forth in claim 11 wherein the formed film is in the form of a thin, absorbable liner component of a cap/collar combination for the femur and further comprising casting a non-absorbable polyurethane composition onto the preformed liner, thereby forming a 2-component intra-articular device comprising a non-absorbable articulating component and an absorbable liner component for intimate contact with defective or diseased femur bone.

13. The method set forth in claim 12 wherein the non-absorbable articulating component of the intra-articular device comprises at least one member of the group consisting of polyether-urethane-urea and polyetherdimethylsiloxane-urethane-urea.

14. A method as in claim 12 wherein the absorbable component of the cap/collar combination is essentially comprises the cap portion of the combination.

15. A solution as in claim 1 further comprising at least one bioactive agent.
